SEQUENCE LISTING (24 sequences)

SEQ ID NO: 1 - protein, 445 aa, Homo sapiens
MPSRAEDYEVLYTIGTGSYGRCQKIRRKSDGKILVWKELDYGSMTEAEKQMLVSEVNLLRELKHPNIVRYYDRIIDRTNTTLYIVMEYCEGGDLASVITKGTKERQYLDEEFVLRVMTQLTLALKECHRRSDGGHTVLHRDLKPANVFLDGKQNVKLGDFGLARILNHDTSFAKTFVGTPYYMSPEQMNRMSYNEKSDIWSLGCLLYELCALMPPFTAFSQKELAGKIREGKFRRIPYRYSDELNEIITRMLNLKDYHRPSVEEILENPLIADLVADEQRRNLERRGRQLGEPEKSQDSSPVLSELKLKEIQLQERERALKAREERLEQKEQELCVRERLAEDKLARAENLLKNYSLLKERKFLSLASNPELLNLPSSVIKKKVHFSGESKENIMRSENSESQLTSKSKCKDLKKRLHAAQLRAQALSDIEKNYQLKSRQILGMR

SEQ ID NO: 2 - DNA, 2051 bp, Homo sapiens
gggcggggttcctggtccctggagctccgcacttggcggcgcaacctgcgtgaggcagcgcgactctggcgactggccggccatgccttcccgggctgaggactatgaagtgttgtacaccattggcacaggctcctacggccgctgccagaagatccggaggaagagtgatggcaagatattagtttggaaagaacttgactatggctccatgacagaagctgagaaacagatgcttgtttctgaagtgaatttgcttcgtgaactgaaacatccaaacatcgttcgttactatgatcggattattgaccggaccaatacaacactgtacattgtaatggaatattgtgaaggaggggatctggctagtgtaattacaaagggaaccaaggaaaggcaatacttagatgaagagtttgttcttcgagtgatgactcagttgactctggccctgaaggaatgccacagacgaagtgatggtggtcataccgtattgcatcgggatctgaaaccagccaatgttttcctggatggcaagcaaaacgtcaagcttggagactttgggctagctagaatattaaaccacgacacgagttttgcaaaaacatttgttggcacaccttattacatgtctcctgaacaaatgaatcgcatgtcctacaatgagaaatcagatatctggtcattgggctgcttgctgtatgagttatgtgcattaatgcctccatttacagcttttagccagaaagaactcgctgggaaaatcagagaaggcaaattcaggcgaattccataccgttactctgatgaattgaatgaaattattacgaggatgttaaacttaaaggattaccatcgaccttctgttgaagaaattcttgagaaccctttaatagcagatttggttgcagacgagcaaagaagaaatcttgagagaagagggcgacaattaggagagccagaaaaatcgcaggattccagccctgtattgagtgagctgaaactgaaggaaattcagttacaggagcgagagcgagctctcaaagcaagagaagaaagattggagcagaaagaacaggagctttgtgttcgtgagagactagcagaggacaaactggctagagcagaaaatctgttgaagaactacagcttgctaaaggaacggaagttcctgtctctggcaagtaatccagaacttcttaatcttccatcctcagtaattaagaagaaagttcatttcagtggggaaagtaaagagaacatcatgaggagtgagaattctgagagtcagctcacatctaagtccaagtgcaaggacctgaagaaaaggcttcacgctgcccagctgcgggctcaagccctgtcagatattgagaaaaattaccaactgaaaagcagacagatcctgggcatgcgctagccaggtagagagacacagagctgtgtacaggatgtaatattaccaacctttaaagactgatattcaaatgctgtagtgttgaatacttggttccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatgttagaaaaaaaatatttatgcaggaaggtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagatatgagctgtctgtcatttacctacttctttcccactaaataaaa

SEQ ID NO: 3 - DNA, 25 bp, Artificial Sequence
  Antisense oligonucleotide
gctcgctctcgctcctgtaactgaa

SEQ ID NO: 4 - DNA, 25 bp, Artificial Sequence
  Antisense oligonucleotide
tcctgcgatttttctggctctccta

SEQ ID NO: 5 - DNA, 25 bp, Artificial Sequence
  Antisense oligonucleotide
ttggctggtttaagatcccgatgca

SEQ ID NO: 6 - DNA, 25 bp, Artificial Sequence
  Antisense oligonucleotide
acggtatgaccaccatcacttcgtc

SEQ ID NO: 7 - DNA, 25 bp, Artificial Sequence
  Antisense oligonucleotide
catacagcaagcagcccaatgacca

SEQ ID NO: 8 - DNA, 25 bp, Artificial Sequence
  Antisense oligonucleotide
accatcacttcgtctgtggcattcc

SEQ ID NO: 9 - DNA, 25 bp, Artificial Sequence
  Antisense oligonucleotide
caacacttcatagtcctcagcccgg

SEQ ID NO: 10 - DNA, 25 bp, Artificial Sequence
  Antisense oligonucleotide
tgctctagccagtttgtcctctgct

SEQ ID NO: 11 - DNA, 25 bp, Artificial Sequence
  Antisense oligonucleotide
gctctcctaattgtcgccctcttct

SEQ ID NO: 12 - DNA, 25 bp, Artificial Sequence
  Antisense oligonucleotide
gacgttttgcttgccatccaggaaa

SEQ ID NO: 13 - DNA, 25 bp, Artificial Sequence
  Reverse control oligonucleotide
aagtcaatgtcctcgctctcgctcg

SEQ ID NO: 14 - DNA, 25 bp, Artificial Sequence
  Reverse control oligonucleotide
atcctctcggtctttttagcgtcct

SEQ ID NO: 15 - DNA, 25 bp, Artificial Sequence
  Reverse control oligonucleotide
acgtagccctagaatttggtcggtt

SEQ ID NO: 16 - DNA, 25 bp, Artificial Sequence
  Reverse control oligonucleotide
ctgcttcactaccaccagtatggca

SEQ ID NO: 17 - DNA, 25 bp, Artificial Sequence
  Reverse control oligonucleotide
accagtaacccgacgaacgacatac

SEQ ID NO: 18 - DNA, 25 bp, Artificial Sequence
  Reverse control oligonucleotide
ccttacggtgtctgcttcactacca

SEQ ID NO: 19 - DNA, 25 bp, Artificial Sequence
  Reverse control oligonucleotide
ggcccgactcctgatacttcacaac

SEQ ID NO: 20 - DNA, 25 bp, Artificial Sequence
  Reverse control oligonucleotide
tcgtctcctgtttgaccgatctcgt

SEQ ID NO: 21 - DNA, 25 bp, Artificial Sequence
  Reverse control oligonucleotide
tcttctcccgctgttaatcctctcg

SEQ ID NO: 22 - DNA, 25 bp, Artificial Sequence
  Reverse control oligonucleotide
aaaggacctaccgttcgttttgcag

SEQ ID NO: 23 - DNA, 1868 bp, Homo sapiens
  misc_feature (1522)..(1522): n is a, c, g, or t
gggcggggttcctggtccctggagctccgcacttggcggcgcaacctgcgtgaggcagcgcgactctggcgactggccggccatgccttcccgggctgaggactatgaagtgttgtacaccattggcacaggctcctacggccgctgccagaagatccggaggaagagtgatggcaagatattagtttggaaagaacttgactatggctccatgacagaagctgagaaacagatgcttgtttctgaagtgaatttgcttcgtgaactgaaacatccaaacatcgttcgttactatgatcggattattgaccggaccaatacaacactgtacattgtaatggaatattgtgaaggaggggatctggctagtgtaattacaaagggaaccaaggaaaggcaatacttagatgaagagtttgttcttcgagtgatgactcagttgactctggccctgaaggaatgccacagacgaagtgatggtggtcataccgtattgcatcgggatctgaaaccagccaatgttttcctggatggcaagcaaaacgtcaagcttggagactttgggctagctagaatattaaaccacgacacgagttttgcaaaaacatttgttggcacaccttattacatgtctcctgaacaaatgaatcgcatgtcctacaatgagaaatcagatatctggtcattgggctgcttgctgtatgagttatgtgcattaatgcctccatttacagcttttagccagaaagaactcgctgggaaaatcagagaaggcaaattcaggcgaattccataccgttactctgatgaattgaatgaaattattacgaggatgttaaacttaaaggattaccatcgaccttctgttgaagaaattcttgagaaccctttaatagcagatttggttgcagacgagcaaagaagaaatcttgagagaagagggcgacaattaggagagccagaaaaatcgcaggattccagccctgtattgagtgagctgaaactgaaggaaattcagttacaggagcgagagcgagctctcaaagcaagagaagaaagattggagcagaaagaacaggagctttgtgttcgtgagagactagcagaggacaaactggctagagcagaaaatctgttgaagaactacagcttgctaaaggaacggaagttcctgtctctggcaagtaatccaggtatgagaatcaacttggtcaacagaagctggtgctacaaatgaagaaatgtgcaccagtgttgctcccaaagtggcttaggtagcccttttcatttacaaatctcaaattttaagatggatttcattgaatatatgcatttcaatggaaacaaaattctgttatagcaatgatttatttcctgggttcatagacctgaggcaatgagtcatagaatttcatgtttgattttctttaccttatcaaactacatgtttagtaattgatacttatttttattatcattatttatttttttattttttttgagatggagtctcactctgtancccaggctggagtgcagtggcacaatctcggctcactgcaagctctgcctccgggttcacgccattcttctgcctcagcctcccaagtagctgggactacagacacccgccaccaagcccggctaatttttgtatttttagtagagacggggtttcaccgtgttagccaggatggtctcgatttcctgacctcgtgatccgcctgcctcggcctcccaatgtgctgggattacaggcatgagacaccgtgcccagccgatacttatttttaaaaattagggcatttacaaaactattgtatgtttcagatttcagagagataaactgcatttcaagtaaaaataaa

SEQ ID NO: 24 - protein, 384 aa, Homo sapiens
MPSRAEDYEVLYTIGTGSYGRCQKIRRKSDGKILVWKELDYGSMTEAEKQMLVSEVNLLRELKHPNIVRYYDRIIDRTNTTLYIVMEYCEGGDLASVITKGTKERQYLDEEFVLRVMTQLTLALKECHRRSDGGHTVLHRDLKPANVFLDGKQNVKLGDFGLARILNHDTSFAKTFVGTPYYMSPEQMNRMSYNEKSDIWSLGCLLYELCALMPPFTAFSQKELAGKIREGKFRRIPYRYSDELNEIITRMLNLKDYHRPSVEEILENPLIADLVADEQRRNLERRGRQLGEPEKSQDSSPVLSELKLKEIQLQERERALKAREERLEQKEQELCVRERLAEDKLARAENLLKNYSLLKERKFLSLASNPGMRINLVNRSWCYK

(19) United States
(12) Patent Application Publication
(10) Pub. No.: US 2004/0019003 A1
(43) Pub. Date: Jan. 29, 2004

(54) Nek2 inhibitors

(75) Inventors: Annette O. Walter, San Carlos, CA (US); Christoph Reinhard, Alameda, CA (US)

Correspondence Address: Chiron Corporation, Intellectual Property, P.O. Box 8097, Emeryville, CA, 94662-8097, US

(73) Assignee: Chiron Corporation, Emeryville, CA

(21) Appl. No.: 10/351,738

(22) Filed: Jan. 24, 2003

Related U.S. Application Data
(60) Provisional application No. 60/352,080, filed on Jan. 24, 2002.

(51) Int. Cl. A61K 48/00; A61K 38/17; A61K 39/395

(52) U.S. Cl. 514/44; 424/145.1; 514/12

(57) ABSTRACT

Inhibitors of human Nek2, including antisense oligonucleotides, methods, and compositions specific for human Nek2, are provided. Methods of using the compositions for modulating Nek2 expression and for regulating cell growth, particularly tumor cell growth, are also provided.

### BACKGROUND OF THE INVENTION

1. Field of the Invention

The present invention provides methods and compositions for modulating the expression and activity of Nek2, and antisense and ribozyme compounds specifically hybridizable with Nek2.

2. Description of the Related Art

Nek2 is a serine/threonine kinase that is structurally related to the mitotic regulator NIMA (“never in mitosis A”). CDC2 (cyclin-dependent kinase) and NIMA kinase may be required for the progression from G2 to mitosis, and these proteins may also be involved in chromatin condensation. WO 99/66051 discloses Nek-related polynucleotide and polypeptide sequences, designated Nek4a (human), Nek4b (human, Nek5 (human), and Nek6 (mouse). WO 00/20448 discloses NLK1-interacting proteins.

Due to its role as a mitotic regulator, with implications for a role in cancer, there is a need in the art for compositions and methods that regulate expression and/or function of Nek2.

### SUMMARY OF THE INVENTION

The present invention provides, in one embodiment, inhibitors of Nek2. Inventive inhibitors include, but are not limited to, antisense molecules, ribozymes, antibodies or antibody fragments, proteins or polypeptides as well as small molecules. Exemplary antisense molecules comprise at least 10, 15 or 20 consecutive nucleotides of or hybridize under stringent conditions to the nucleic acid of SEQ ID NO:1 or 23. More preferred are antisense molecules that comprise at least 25 consecutive nucleotides of or hybridize under stringent conditions to the sequence of SEQ ID NO:1 or 23. Representative antisense molecules are provided herein as SEQ ID NOs:3-12.

In further embodiments, compositions are provided that comprise one or more Nek2 inhibitor in a pharmaceutically acceptable carrier.

Additional embodiments provide methods of decreasing Nek2 gene expression or biological activity.

The invention provides an antisense oligonucleotide comprising at least one modified internucleoside linkage.

The invention further provides an antisense oligonucleotide having a phosphorothioate linkage.

The invention still further provides an antisense oligonucleotide comprising at least one modified sugar moiety.

The invention also provides an antisense oligonucleotide comprising at least one modified sugar moiety which is a 2′-O-methyl sugar moiety.

The invention further provides an antisense oligonucleotide comprising at least one modified nucleobase.

The invention still further provides an antisense oligonucleotide having a modified nucleobase wherein the modified nucleobase is 5-methylcytosine.

The invention also provides an antisense compound wherein the antisense compound is a chimeric oligonucleotide.

The invention provides a method of inhibiting the expression of human Nek2 in human cells or tissues comprising contacting the cells or tissues in vivo with an antisense compound or a ribozyme of 8 to 35 nucleotides in length targeted to a nucleic acid molecule encoding human Nek2 so that expression of human Nek2 is inhibited.

The invention further provides a method of modulating growth of cancer cells comprising contacting the cancer cells in vivo with an antisense compound or ribozyme of 8 to 35 nucleotides in length targeted to a nucleic acid molecule encoding human Nek2 so that expression of human Nek2 is inhibited.

The invention still further provides for identifying target regions of Nek2 polynucleotides. The invention also provides labeled probes for identifying Nek2 polynucleotides by in situ hybridization.

The invention provides for the use of an Nek2 inhibitor according to the invention to prepare a medicament for modulating cell proliferation.

The invention also provides a pharmaceutical composition for inhibiting expression of the Nek2, comprising an antisense oligonucleotide according to the invention in a mixture with a physiologically acceptable carrier or diluent.

The invention further provides a ribozyme capable of specifically cleaving Nek2 RNA, and a pharmaceutical composition comprising the ribozyme.

The invention also provides a method of identifying small molecule inhibitors of Nek2 wherein the inhibitors are capable of reducing the activity of Nek2 or of reducing or preventing the expression of Nek2 mRNA.

The invention therefore provides an isolated Nek2 inhibitor selected from the group consisting of an antisense oligonucleotide, a ribozyme, a protein, a polypeptide, an antibody, and a small molecule.

In a specific embodiment, the isolated Nek2 inhibitor is an antisense molecule.

In a more specific embodiment, the isolated Nek2 inhibitor antisense molecule or the complement thereof comprises at least 10 consecutive nucleic acids of the sequence of SEQ ID NO:1 or 23.

In another specific embodiment, the isolated Nek2 inhibitor antisense molecule or the complement thereof hybridizes under high stringency conditions to the sequence of SEQ ID NO:1 or 23.

In specific embodiments, the isolated Nek2 inhibitor antisense molecule comprises a nucleic acid sequence selected from the group consisting of SEQ ID NOs:3-12.

In another embodiment, the isolated Nek2 inhibitor is a ribozyme, and in yet other embodiments, the isolated Nek2 inhibitor is selected from the group consisting of an antibody and an antibody fragment.

The invention further provides a composition comprising a therapeutically effective amount of a Nek2 inhibitor in a pharmaceutically acceptable carrier.

In certain embodiments, the composition comprises two or more Nek2 inhibitors in the composition, and the Nek2 inhibitor is an antisense molecule.

In specific embodiments of the composition, the antisense molecule or the complement thereof comprises at least 10 consecutive nucleic acids of the sequence of SEQ ID NO:1 or 23, and in more specific embodiments of the composition, the antisense molecule comprises a nucleic acid sequence selected from the group consisting of SEQ ID NOs:3-12.

The invention yet further provides a method of inhibiting the expression of Nek2 in a mammalian cell, comprising administering to said cell an Nek2 inhibitor selected from the group consisting of an antisense oligonucleotide, a ribozyme, a protein, a polypeptide, an antibody, and a small molecule.

In certain embodiments of this method, the Nek2 inhibitor is an antisense molecule.

The invention still further provides a method of inhibiting the expression of Nek2 gene expression in a subject, comprising administering to said subject, in a pharmaceutically effective vehicle, an amount of an antisense oligonucleotide which is effective to specifically hybridize to all or part of a selected target nucleic acid sequence derived from said Nek2 gene.

In certain embodiments of this method, the antisense oligonucleotide is selected from the group consisting of SEQ ID NOs:3-12.

The invention also provides a method of treating neoplastic disease, comprising administering to a mammalian cell an Nek2 inhibitor selected from group consisting of an antisense oligonucleotide, a ribozyme, a protein, a polypeptide, an antibody, and a small molecule such that the neoplastic disease is reduced in severity.

Other embodiments provide an antisense compound of 8 to 35 nucleotides in length targeted to a nucleic acid molecule encoding human Nek2, wherein the antisense compound inhibits the expression of human Nek2, and an isolated polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOs:3-12.

Also provided is a recombinant vector comprising a polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOs:3-12.

### BRIEF DESCRIPTION OF THE DRAWINGS

FIG. 1 is a Nek2 polynucleotide (SEQ ID NO:2).

FIG. 2 is a Nek2 polypeptide (SEQ ID NO:1).

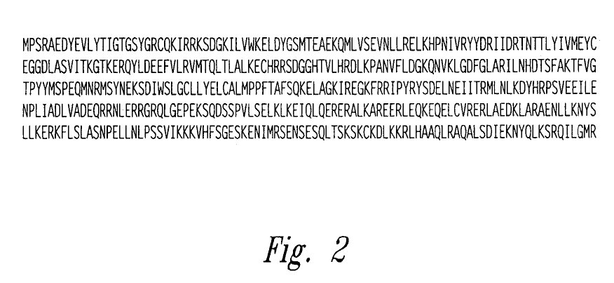

FIG. 3 is a bar graph showing Nek2 messenger RNA (C103) levels in cell lines, normalized to actin.

FIG. 4 depicts two graphs showing that Nek2 antisense (SEQ ID NO:12) led to a decrease in cell proliferation in SW620 cells (FIG. 4A) and normal fibroblasts (MRC9 cells) (FIG. 4B). Wt: untreated; AS: SEQ ID NO:12 (in duplicate); RC: SEQ ID NO:21 (control, in duplicate).

FIG. 5 is a bar graph showing that Nek2 antisense oligonucleotide treatment induces cytotoxicity, as measured by LDH assay.

FIG. 6 is a bar graph showing cell death induced by Nek2 antisense in normal fibroblasts.

FIG. 7 is a bar graph showing the effect of Nek2 depletion on anchorage independent growth.

FIG. 8 shows that an anti-Nek2 antibody detects Nek2 protein in untreated SW620 cells, and that the amount of protein detected is reduced at 24 hours in SW620 cells treated with SEQ ID NO:12; there is less reduction in protein in cells treated with the reverse complement, SEQ ID NO:21. The figure also shows that HIS-tag Nek2 can be detected in Sf9 insect cells transfected with Nek2 encoding polynucleotide.

### DETAILED DESCRIPTION OF THE INVENTION

Introduction

The invention relates to the use of inhibitors, preferably oligonucleotides, such as antisense molecules or ribozymes, to target and modulate the expression of polynucleotides comprising an Nek2 nucleotide sequence. Nek2 is a protein kinase, a member of a family of serine/threonine kinases structurally related to the mitotic regulator NIMA of aspergillus nidulans.

Nek2 mRNA levels are elevated in 43% of breast cancer samples compared to normal tissue. Inhibition of Nek2 expression through treatment with Nek2 antisense oligos suppressed anchorage-independent growth of tumor cells transfected with Nek2 antisense oligonucleotides. Nek2 antisense oligonucleotides also inhibited cell proliferation and induced release of lactate dehydrogenase, indicating cell death. Taken together, these results indicate that Nek2 is an important target for inhibiting tumor cell growth.

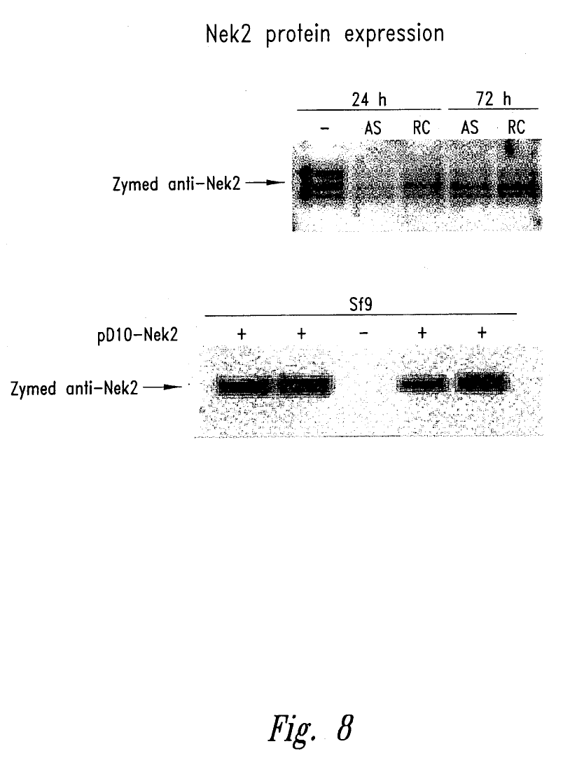

Ten antisense oligonucleotides were prepared, and are disclosed herein as SEQ ID NOs:3-12. When transfected into SW620 cells, each antisense oligonucleotide caused a reduction in Nek2 message levels. SEQ ID NO:12 was selected for further studies of the effect on cell growth, anchorage-dependent growth, and Nek2 protein expression. Protein expression was detected using a Zymed anti-Nek2 antibody. 24 hours after transfection with the oligonucleotide of SEQ ID NO:12, the Nek2 protein levels decreased significantly, with some recovery at 72 hours (FIG. 8). The reverse control oligonucleotide did not significantly reduce Nek2 protein expression.

Oligonucleotides for Targeting Nek2 Polynucleotides

According to the present invention, oligonucleotide molecules capable of hybridizing with Nek2 polynucleotides inhibited the proliferation of ovarian and colon cancer cell lines. These cell lines are standard models for cancer cell proliferation and growth in vivo, and the results support in vivo use of the Nek2 antisense molecules to ameliorate cancer in humans and other mammals. The SW620 cell line is an established model system for colon cancer.

The present invention relates to antisense oligonucleotides directed to Nek2 polynucleotides. Transfecting SW620 cells with a plasmid encoding antisense FasL cDNA suggests that impairing FasL translation can inhibit tumor progression (Nyhus et al., Gene Ther. (2001) 8:209-14). p53 antisense oligonucleotides inhibited the growth of SW620 (Hirota et al., Jpn. J Cancer Res. (1996) 87:635-42), and reducing matrilysin levels in SW620 cells through antisense treatment has been suggested as a method for reducing tumorigenicity and progression of colorectal tumors (Witty et al., Cancer Res. (1994) 54:4805-12). The present invention adds to this knowledge about treating cancer cells by disclosing for the first time that antisense oligonucleotides directed against Nek2 are suitable agents for treating breast cancer.

Included within the scope of the invention are oligonucleotides capable of hybridizing with Nek2 DNA or RNA, referred to as the target polynucleotide. An oligonucleotide need not be 100% complementary to the target polynucleotide, as long as specific hybridization is achieved. The degree of hybridization to be achieved is that which interferes with the normal function of the target polynucleotide, be it transcription, translation, pairing with a complementary sequence, or binding with another biological component such as a protein. An antisense oligonucleotide can interfere with DNA replication and transcription, and it can interfere with RNA translocation, translation, splicing, and catalytic activity.

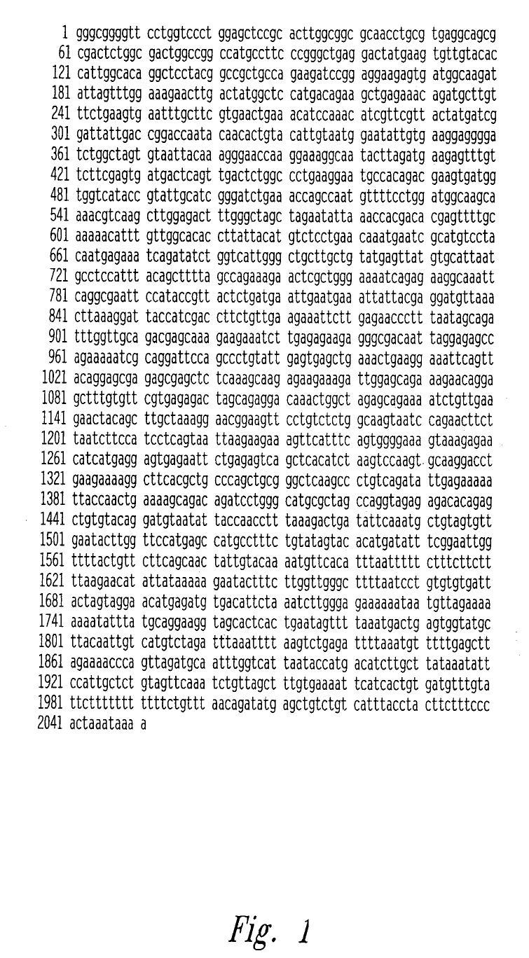

The invention includes within its scope any oligonucleotide of about 8 to about 35 nucleotides in length, including variations as described herein, wherein the oligonucleotide hybridizes to a Nek2 polynucleotide, including DNA or mRNA, such that an effect on the normal function of the polynucleotide is achieved. The oligonuclelotide can be 8, 10, 15, 17, 18, 20, 22, 25, 28, 30, 32 or 35 nucleotides in length. The nucleotide sequence of Nek2 is shown in FIG. 1 (SEQ ID NO:2), and a splice variant (Genbank AY045701) is shown in SEQ ID NO:23. The corresponding translated polypeptide is shown in SEQ ID NO:24. Preferred antisense oligonucleotides include:

| | | |
|---|---|---|
| CHIR-103-1 | GCTCGCTCTCGCTCCTGTAACTGAA | SEQ ID NO:3 |
| CHIR-103-2 | TCCTGCGATTTTTCTGGCTCTCCTA | SEQ ID NO:4 |
| CHIR-103-3 | TTGGCTGGTTTAAGATCCCGATGCA | SEQ ID NO:5 |
| CHIR-103-4 | ACGGTATGACCACCATCACTTCGTC | SEQ ID NO:6 |
| CHIR-103-5 | CATACAGCAAGCAGCCCAATGACCA | SEQ ID NO:7 |
| CHIR-103-6 | ACCATCACTTCGTCTGTGGCATTCC | SEQ ID NO:8 |
| CHIR-103-7 | CAACACTTCATAGTCCTCAGCCCGG | SEQ ID NO:9 |
| CHIR-103-8 | TGCTCTAGCCAGTTTGTCCTCTGCT | SEQ ID NO:10 |
| CHIR-103-9 | GCTCTCCTAATTGTCGCCCTCTTCT | SEQ ID NO:11 |
| CHIR-103-10 | GACGTTTTGCTTGCCATCCAGGAAA | SEQ ID NO:12 |

The antitumor use of the oligonucleotides disclosed herein is based on the discovery that Nek2 antisense oligonucleotides can reduce Nek2 mRNA levels in tumor cells, and can inhibit proliferation of tumor cells. To measure the effect on mRNA, tumor cells were incubated with a transfection mixture of an oligonucleotide and a carrier, specifically a lipitoid or cholesteroid, although other carriers can be used as is known in the art. After an incubation of 2-24 hours, the transfection mixture was removed and replaced with normal growth media as described in the Examples.

Total RNA was extracted from the cells, reverse transcribed, and amplified as described in the Examples. Transfection of the cells with any one of the antisense oligonucleotides (SEQ ID NOs:3-12) reduced the Nek2 mRNA levels relative to actin.

Soft agar assays were performed to determine the effect of the antisense oligonucleotides on anchorage-dependent growth. In both cell lines, colony formation was inhibited by each antisense oligonucleotide. This result demonstrates that inhibition of Nek2 expression also inhibits the ability of tumor cells to grow in an anchorage-independent manner.

Examples of preferred antisense compounds useful in the invention are based on SEQ ID NOs:3-12, and include oligonucleotides containing modified backbones or non-natural internucleoside linkages. Oligonucleotides having modified backbones include those retaining a phosphorus atom in the backbone, and those that do not have a phosphorus atom in the backbone. Preferred modified oligonucleotide backbones include phosphorothioates, chiral phosphorothioates, phosphotriesters, aminoalkylphosphotriesters, methyl and other alkyl phosphonates including 3′-alkylene phosphonates and chiral phosphonates, phosphinates, phosphoroamidates including 3′-amino phosphoroamidate and aminoalkylphosphoroamidates, thiophosphoroamidates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′5′ to 5′-2′. Various salts, mixed salts and free acid forms are also included.

Examples of 20-mer oligonucleotides include the following oligonucleotides, indicated by polynucleotide positions with reference to SEQ ID NO:1: 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512,494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567,549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587,569-588, 570-589, 571-590, 572-591,573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037,1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051,1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082,1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377,1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, 1849-1868, 1850-1869, 1851-1870, 1852-1871, 1853-1872, 1854-1873, 1855-1874, 1856-1875, 1857-1876, 1858-1877, 1859-1878, 1860-1879, 1861-1880, 1862-1881, 1863-1882, 1864-1883, 1865-1884, 1866-1885, 1867-1886, 1868-1887, 1869-1888, 1870-1889, 1871-1890, 1872-1891, 1873-1892, 1874-1893, 1875-1894, 1876-1895, 1877-1896, 1878-1897, 1879-1898, 1880-1899, 1881-1900, 1882-1901, 1883-1902, 1884-1903, 1885-1904, 1886-1905, 1887-1906, 1888-1907, 1889-1908, 1890-1909, 1891-1910, 1892-1911, 1893-1912, 1894-1913, 1895-1914, 1896-1915, 1897-1916, 1898-1917, 1899-1918, 1900-1919, 1901-1920, 1902-1921, 1903-1922, 1904-1923, 1905-1924, 1906-1925, 1907-1926, 1908-1927, 1909-1928, 1910-1929, 1911-1930, 1912-1931, 1913-1932, 1914-1933, 1915-1934, 1916-1935, 1917-1936, 1918-1937, 1919-1938, 1920-1939, 1921-1940, 1922-1941, 1923-1942, 1924-1943, 1925-1944, 1926-1945, 1927-1946, 1928-1947, 1929-1948, 1930-1949, 1931-1950, 1932-1951, 1933-1952, 1934-1953, 1935-1954, 1936-1955, 1937-1956, 1938-1957, 1939-1958, 1940-1959, 1941-1960, 1942-1961, 1943-1962, 1944-1963, 1945-1964, 1946-1965, 1947-1966, 1948-1967, 1949-1968, 1950-1969, 1951-1970, 1952-1971, 1953-1972, 1954-1973, 1955-1974, 1956-1975, 1957-1976, 1958-1977, 1959-1978, 1960-1979, 1961-1980, 1962-1981, 1963-1982, 1964-1983, 1965-1984, 1966-1985, 1967-1986, 1968-1987, 1969-1988, 1970-1989, 1971-1990, 1972-1991, 1973-1992, 1974-1993, 1975-1994, 1976-1995, 1977-1996, 1978-1997, 1979-1998, 1980-1999, 1981-2000, 1982-2001, 1983-2002, 1984-2003, 1985-2004, 1986-2005, 1987-2006, 1988-2007, 1989-2008, 1990-2009, 1991-2010, 1992-2011, 1993-2012, 1994-2013, 1995-2014, 1996-2015, 1997-2016, 1998-2017, 1999-2018, 2000-2019, 2001-2020, 2002-2021, 2003-2022, 2004-2023, 2005-2024, 2006-2025, 2007-2026, 2008-2027, 2009-2028, 2010-2029, 2011-2030, 2012-2031, 2013-2032, 2014-2033, 2015-2034, 2016-2035, 2017-2036, 2018-2037, 2019-2038, 2020-2039, 2021-2040, 2022-2041, 2023-2042, 2024-2043, 2025-2044, 2026-2045, 2027-2046, 2028-2047, 20292048, 2030-2049, 2031-2050, and 2032-2051.

Examples of 25-mer oligonucleotides include the following oligonucleotides, indicated by polynucleotide positions with reference to SEQ ID NO:1: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176,153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316,.293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487,464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820,797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992,969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-l1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568,1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, 1844-1868, 1845-1869, 1846-1870, 1847-1871, 1848-1872, 1849-1873, 1850-1874, 1851-1875, 1852-1876, 1853-1877, 1854-1878, 1855-1879, 1856-1880, 1857-1881, 1858-1882, 1859-1883, 1860-1884, 1861-1885, 1862-1886, 1863-1887, 1864-1888, 1865-1889, 1866-1890, 1867-1891, 1868-1892, 1869-1893, 1870-1894, 1871-1895, 1872-1896, 1873-1897, 1874-1898, 1875-1899, 1876-1900, 1877-1901, 1878-1902, 1879-1903, 1880-1904, 1881-1905, 1882-1906, 1883-1907, 1884-1908, 1885-1909, 1886-1910, 1887-1911, 1888-1912, 1889-1913, 1890-1914, 1891-1915, 1892-1916, 1893-1917, 1894-1918, 1895-1919, 1896-1920, 1897-1921, 1898-1922, 1899-1923, 1900-1924, 1901-1925, 1902-1926, 1903-1927, 1904-1928, 1905-1929, 1906-1930, 1907-1931, 1908-1932, 1909-1933, 1910-1934, 1911-1935, 1912-1936, 1913-1937, 1914-1938, 1915-1939, 1916-1940, 1917-1941, 1918-1942, 1919-1943, 1920-1944, 1921-1945, 1922-1946, 1923-1947, 1924-1948, 1925-1949, 1926-1950, 1927-1951, 1928-1952, 1929-1953, 1930-1954, 1931-1955, 1932-1956, 1933-1957, 1934-1958, 1935-1959, 1936-1960, 1937-1961, 1938-1962, 1939-1963, 1940-1964, 1941-1965, 1942-1966, 1943-1967, 1944-1968, 1945-1969, 1946-1970, 1947-1971, 1948-1972, 1949-1973, 1950-1974, 1951-1975, 1952-1976, 1953-1977, 1954-1978, 1955-1979, 1956-1980, 1957-1981, 1958-1982, 1959-1983, 1960-1984, 1961-1985, 1962-1986, 1963-1987, 1964-1988, 1965-1989, 1966-1990, 1967-1991, 1968-1992, 1969-1993, 1970-1994, 1971-1995, 1972-1996, 1973-1997, 1974-1998, 1975-1999, 1976-2000, 1977-2001, 1978-2002, 1979-2003, 1980-2004, 1981-2005, 1982-2006, 1983-2007, 1984-2008, 1985-2009, 1986-2010, 1987-2011, 1988-2012, 1989-2013, 1990-2014, 1991-2015, 1992-2016, 1993-2017, 1994-2018, 1995-2019, 1996-2020, 1997-2021, 1998-2022, 1999-2023, 2000-2024, 2001-2025, 2002-2026, 2003-2027, 2004-2028, 2005-2029, 2006-2030, 2007-2031, 2008-2032, 2009-2033, 2010-2034, 2011-2035, 2012-2036, 2013-2037, 2014-2038, 2015-2039, 2016-2040, 2017-2041, 2018-2042, 2019-2043, 2020-2044, 2021-2045, 2022-2046, 2023-2047, 2024-2048, 2025-2049, 2026-2050, and 2027-2051.

Examples of additional 20-mer oligonucleotides include the following oligonucleotides, indicated by polynucleotide positions with reference to SEQ ID NO:23: 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439,421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1.754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, and 1849-1868.

Examples of additional 25-mer oligonucleotides include the following oligonucleotides, indicated by polynucleotide positions with reference to SEQ ID NO:23: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815,792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, and 1844-1868.

The present invention relates to antisense oligonucleotides designed to interfere with the normal function of Nek2 polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Preferred antisense oligonucleotides in addition to those of SEQ ID NOs:3-12 can be selected by routine experimentation using, for example, assays described in the Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles, R. V. et al., Nuc. Acids Res. (1995) 23:954-961; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

As disclosed in U.S. Pat. No. 5,998,383, incorporated herein by reference, the oligonucleotide is selected such that the sequence exhibits suitable energy related characteristics important for oligonucleotide duplex formation with their complementary templates, and shows a low potential for self-dimerization or self-complementation (Anazodo et al., Biochem. Biophys. Res. Commun. (1996) 229:305-309). The computer program OLIGO (Primer Analysis Software, Version 3.4), is used to determined antisense sequence melting temperature, free energy properties, and to estimate potential self-dimer formation and self-complimentarity properties. The program allows the determination of a qualitative estimation of these two parameters (potential self-dimer formation and self-complimentary) and provides an indication of “no potential” or “some potential” or “essentially complete potential.” Segments of Nek2 polynucleotides are generally selected that have estimates of no potential in these parameters. However, segments can be used that have “some potential” in one of the categories. A balance of the parameters is used in the selection.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in Example 1. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. (1998) 23:45-50.) According to the present invention, cultures of SW620 cells were transfected with five different antisense oligonucleotides designed to target Nek2 phosphatase. These oligonucleotides are shown in SEQ ID NOs:3-12. The levels of mRNA corresponding to Nek2 were measured in treated and control cells. SEQ ID NOs:3-12 caused dramatic decreases in Nek2 mRNA when normalized to actin mRNA levels.

Additional inhibitors include RNAi, ribozymes, proteins or polypeptides, antibodies or fragments thereof as well as small molecules. Each of these Nek2 inhibitors share the common feature in that they reduce the expression and/or biological activity of Nek2. In addition to the exemplary Nek2 inhibitors disclosed herein, alternative inhibitors may be obtained through routine experimentation utilizing methodology either specifically disclosed herein or as otherwise readily available to and within the expertise of the skilled artisan.

RNAi

The invention also contemplates introduction of RNA with partial or fully double-stranded character into the cell or into the extracellular environment. Inhibition is specific to the Nek2 expression in that a nucleotide sequence from a portion of the target Nek2 gene is chosen to produce inhibitory RNA. This process is (1) effective in producing inhibition of gene expression, and (2) specific to the targeted NEK2 gene. The procedure may provide partial or complete loss of function for the target Nek2 gene. A reduction or loss of gene expression in at least 99% of targeted cells has been shown. Lower doses of injected material and longer times after administration of dsRNA may result in inhibition in a smaller fraction of cells. Quantitation of gene expression in a cell may show similar amounts of inhibition at the level of accumulation of target mRNA or translation of target protein. Methods of preparing and using RNAi are generally disclosed in U.S. Pat. No. 6,506,559, incorporated herein by reference.

The RNA may comprise one or more strands of polymerized ribonucleotide; it may include modifications to either the phosphate-sugar backbone or the nucleoside. The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequences identical to a portion of the Nek2 target gene is preferred for inhibition. RNA sequences with insertions, deletions, and single point mutations relative to the target sequence have also been found to be effective for inhibition. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For RNAi, the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected. Physical methods of introducing nucleic acids include injection directly into the cell or extracellular injection into the organism of an RNA solution.

The advantages of the method include: the ease of introducing double-stranded RNA into cells, the low concentration of RNA which can be used, the stability of double-stranded RNA, and the effectiveness of the inhibition.

Inhibition of gene expression refers to the absence (or observable decrease) in the level of protein and/or mRNA product from a Nek2 target gene. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of dsRNA may result in inhibition in a smaller fraction of cells (e.g., at least 10%, 20%, 50%, 75%, 90%, or 95% of targeted cells). Quantitation of Nek2 gene expression in a cell may show similar amounts of inhibition at the level of accumulation of Nek2 target mRNA or translation of Nek2 target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

The RNA may comprise one or more strands of polymerized ribonucleotide. It may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

RNA containing a nucleotide sequences identical to a portion of the Nek2 target gene are preferred for inhibition. RNA sequences with insertions, deletions, and single point mutations relative to the target sequence may be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the Nek2 target gene is preferred. Alternatively, the duplex region of the RNA may be defined functionally as a nucleotide sequence that is capable of hybridizing with a portion of the Nek2 target gene transcript (e.g., 400 mM NaCl, 40 mM PIPES pH 6.4, 1 mM EDTA, 50.degree. C. or 70.degree. C. hybridization for 12-16 hours; followed by washing). The length of the identical nucleotide sequences may be at least 25, 50, 100, 200, 300 or 400 bases.

100% sequence identity between the RNA and the Nek2 target gene is not required to practice the present invention. Thus the methods have the advantage of being able to tolerate sequence variations that might be expected due to genetic mutation, strain polymorphism, or evolutionary divergence.

Nek2 RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art. (see also (for example, WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

RNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The present invention may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

Ribozymes

Nek2 inhibitors may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell (1987) 48:211-220; Haseloff and Gerlach, Nature (1988) 328:596-600; Walbot and Bruening, Nature (1988) 334:196; Haseloff and Gerlach, Nature (1988) 334:585); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such Nek2 mRNA-specific ribozyme, or a nucleic acid encoding such a ribozyme, may be delivered to a host cell to effect inhibition of Nek2 gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed.

Proteins and Polypeptides

In addition to the antisense molecules and ribozymes disclosed herein, Nek2 inhibitors of the present invention also include proteins or polypeptides that are effective in either reducing Nek2 gene expression or in decreasing one or more of Nek2's biological activities. A variety of methods are readily available in the art by which the skilled artisan may, through routine experimentation, rapidly identify such Nek2 inhibitors. The present invention is not limited by the following exemplary methodologies.

Inhibitors of Nek2's biological activities encompass those proteins and/or polypeptides that interfere with Nek2's dephosphorylation activity. Such interference may occur through direct interaction with Nek2's active domain or indirectly through non- or un-competitive inhibition such as via binding to an allosteric site. Accordingly, available methods for identifying proteins and/or polypeptides that bind to Nek2 may be employed to identify lead compounds that may, through the methodology disclosed herein, be characterized for their Nek2 inhibitory activity.

Literature is available to the skilled artisan that describes methods for detecting and analyzing protein-protein interactions. Reviewed in Phizicky, E. M. et al., Microbiological Reviews (1995) 59:94-123 incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive Nek2 inhibitors may be identified through biological screening assays that rely on the direct interaction between the Nek2 protein and a panel or library of potential inhibitor proteins. Biological screening methodologies, including the various “n-hybrid technologies,” are described in, for example, Vidal, M. et al., Nucl. Acids Res. (1999) 27(4):919-929; Frederickson, R. M., Curr Opin. Biotechnol. (1998) 9(1):90-6; Brachmann, R. K. et al., Curr. Opin. Biotechnol. (1997) 8(5):561-568; and White, M. A., Proc. Natl. Acad. Sci. U.S.A. (1996) 93:10001-10003 each of which is incorporated herein by reference.

The two-hybrid screening methodology may be employed to search new or existing target cDNA libraries for Nek2 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C.-T. et al., Proc. Natl. Acad. Sci. U.S.A. (1991) 88:9578-9582; Dalton, S. et al., Cell (1992) 68:597-612; Durfee, T. K. et al., Genes Dev. (1993) 7:555-569; Vojtek, A. B. et al., Cell (1993) 74:205-214; and Zervos, A. S. et al., Cell (1993) 72:223-232. Commonly used reporter genes include the E. coli lacZ gene as well as selectable yeast genes such as HIS3 and LEU2. Fields, S. et al., Nature (London) (1989) 340:245-246; Durfee, T. K., supra; and Zervos, A. S., supra. A wide variety of activation domain libraries is readily available in the art such that the screening for interacting proteins may be performed through routine experimentation.

Suitable bait proteins for the identification of Nek2 interacting proteins may be designed based on the Nek2 cDNA sequence presented herein as SEQ ID NO:1. Such bait proteins include either the full-length Nek2 protein or fragments thereof.

Plasmid vectors, such as, e.g., pBTM116 and pAS2-1, for preparing Nek2 bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

Nek2 inhibitors of the present invention may alternatively be identified through one of the physical or biochemical methods available in the art for detecting protein-protein interactions.

Through the protein affinity chromatography methodology, lead compounds to be tested as potential Nek2 inhibitors may be identified by virtue of their specific retention to Nek2 when either covalently or non-covalently coupled to a solid matrix such as, e.g., Sepharose beads. The preparation of protein affinity columns is described in, for example, Beeckmans, S. et al., Eur. J. Biochem. (1981) 117:527-535 and Formosa, T. et al., Methods Enzymol. (1991) 208:24-45. Cell lysates containing the full complement of cellular proteins may be passed through the Nek2 affinity column. Proteins having a high affinity for Nek2 will be specifically retained under low-salt conditions while the majority of cellular proteins will pass through the column. Such high affinity proteins may be eluted from the immobilized Nek2 under conditions of high-salt, with chaotropic solvents or with sodium dodecyl sulfate (SDS). In some embodiments, it may be preferred to radiolabel the cells prior to preparing the lysate as an aid in identifying the Nek2 specific binding proteins. Methods for radiolabeling mammalian cells are well known in the art and are provided, e.g., in Sopta, M. et al., J. Biol. Chem. (1985) 260:10353-10360.

Suitable Nek2 proteins for affinity chromatography may be fused to a protein or polypeptide to permit rapid purification on an appropriate affinity resin. For example, the Nek2 cDNA may be fused to the coding region for glutathione S-transferase (GST) which facilitates the adsorption of fusion proteins to glutathione-agarose columns. Smith et al., Gene (1988) 67:31-40. Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of Nek2 fusion proteins that is presented herein is the epitope for the influenza virus hemagglutinin (HA) against which monoclonal antibodies are readily available and from which antibodies an affinity column may be prepared.

Proteins that are specifically retained on a Nek2 affinity column may be identified after subjecting to SDS polyacrylamide gel electrophoresis (SDS-PAGE). Thus, where cells are radiolabeled prior to the preparation of cell lysates and passage through the Nek2 affinity column, proteins having high affinity for Nek2 may be detected by autoradiography. The identity of Nek2 specific binding proteins may be determined by protein sequencing techniques that are readily available to the skilled artisan, such as Mathews, C. K. et al., Biochemistry, The Benjamin/Cummings Publishing Company, Inc. pp. 166-170 (1990).

Antibodies or Antibody Fragments

Nek2 inhibitors of the present invention include antibodies and/or antibody fragments that are effective in reducing Nek2 gene expression and/or biological activity. Suitable antibodies may be monoclonal, polyclonal or humanized monoclonal antibodies. Antibodies may be derived by conventional hybridoma based methodology, from antisera isolated from Nek2 inoculated animals or through recombinant DNA technology. Alternatively, inventive antibodies or antibody fragments may be identified in vitro by use of one or more of the readily available phage display libraries. Exemplary methods are disclosed herein.

In one embodiment of the present invention, Nek2 inhibitors are monoclonal antibodies that may be produced as follows. Nek2 protein may be produced, for example, by expression of Nek2 cDNA in a baculovirus based system. By this method, Nek2 cDNA or a fragment thereof is ligated into a suitable plasmid vector that is subsequently used to transfect Sf9 cells to facilitate protein production. In addition, it may be advantageous to incorporate an epitope tag or other moiety to facilitate affinity purification of the Nek2 protein. According to the invention, His-tagged Nek2 has been expressed in Sf9 cells, demonstrating the feasibility of this method as applied to Nek2. Clones of Sf9 cells expressing Nek2 are identified, e.g., by enzyme linked immunosorbant assay (ELISA), lysates are prepared and the Nek2 protein purified by affinity chromatography and the purified protein is injected, intraperitoneally, into BALB/c mice to induce antibody production. It may be advantageous to add an adjuvant, such as Freund's adjuvant, to increase the resulting immune response.

Serum is tested for the production of specific antibodies and spleen cells from animals having a positive specific antibody titer are used for cell fusions with myeloma cells to generate hybridoma clones. Supernatants derived from hybridoma clones are tested for the presence of monoclonal antibodies having specificity against Nek2. For a general description of monoclonal antibody methodology, See, e.g., Harlow and Lane, Antibodies: A Laboratory Manual, Cold Spring Harbor Laboratory (1988).

In addition to the baculovirus expression system, other suitable bacterial or yeast expression systems may be employed for the expression of Nek2 protein or polypeptides thereof. As with the baculovirus system, it may be advantageous to utilize one of the commercially available affinity tags to facilitate purification prior to inoculation of the animals. Thus, the Nek2 cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In other embodiments of the present invention, Nek2 inhibitors are humanized anti-Nek2 monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature (1986) 321:522-525; Morrison et al., Proc. Natl. Acad. Sci., U.S.A., (1984) 81:6851-6855; Morrison and Oi, Adv. Immunol. (1988) 44:65-92; Verhoeyer et al., Science (1988) 239:1534-1536; Padlan, Molec. Immun. (1991) 28:489-498; Padlan, Molec. Immunol. (1994) 31(3):169-217; and Kettleborough, C. A. et al., Protein Eng. (1991) 4:773-83 each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. (1987) 196:901-917; Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Humanized antibodies to Nek2 can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFα, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, Nek2 polypeptides of the invention and variants thereof are used to immunize a transgenic animal as described above. Monoclonal antibodies are made using methods known in the art, and the specificity of the antibodies is tested using isolated Nek2 polypeptides. The suitability of the antibodies for clinical use is tested by, for example, exposing SW620 cells to the antibodies and measuring cell growth. According to the invention, inhibition of Nek2 expression using antisense oligonucleotides specific for Nek2 polynucleotides causes an inhibition of anchorage-independent growth of a colon cancer cell line, SW620. The antisense oligonucleotides also inhibited the proliferation of a ovarian cancer cell line, SKOV3. Human monoclonal antibodies specific for Nek2 or a variant or fragment thereof can be tested for their ability to inhibit proliferation, colony growth, or any other biological parameter indicative of control of tumor growth, migration, or metastasis, particularly tumor cells of epithelial origin. Such antibodies would be suitable for pre-clinical and clinical trials as pharmaceutical agents for preventing or controlling growth of cancer cells.

It will be appreciated that alternative Nek2 inhibitor antibodies may be readily obtained by other methods commonly known in the art. One exemplary methodology for identifying antibodies having a high specificity for Nek2 is the phage display technology.

Phage display libraries for the production of high-affinity antibodies are described in, for example, Hoogenboom, H. R et al., Immunotechnology (1998) 4(1):1-20; Hoogenboom, H. R., Trends Biotechnol. (1997) 15:62-70 and McGuinness, B. et al., Nature Bio. Technol. (1996) 14:1149-1154 each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature (1990) 348:552-554 which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

Nek2 protein suitable for screening a phage library may be obtained by, for example, expression in baculovirus Sf9 cells as described, supra. Alternatively, the Nek2 coding region may be PCR amplified using primers specific to the desired region of the Nek2 protein. As discussed above, the Nek2 protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-Nek2 binding properties may subsequently be isolated by successive panning, in the case of a solid matrix, or by affinity adsorption to a Nek2 antigen column. Phage having the desired Nek2 inhibitory activities may be reintroduced into bacteria by infection and propagated by standard methods known to those skilled in the art. See Hoogenboom, H. R., Trends Biotechnol., supra for a review of methods for screening for positive antibody-pIII phage.

Small Molecules

The present invention also provides small molecule Nek2 inhibitors that may be readily identified through routine application of high-throughput screening (HTS) methodologies. Reviewed by Persidis, A., Nature Biotechnology (1998) 16:488-489. HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. See, e.g., Gonzalez, J. E. et al., Curr. Opin. Biotechnol. (1998) 9(6):624-631 incorporated herein by reference.

HTS methodology may be employed, e.g., to screen for lead compounds that block one of Nek2's biological activities. By this method, Nek2 protein may be immunoprecipitated from cells expressing the protein and applied to wells on an assay plate suitable for robotic screening. Individual test compounds may then be contacted with the immunoprecipitated protein and the effect of each test compound on Nek2 kinase activity assessed by, e.g., incubating in the presence of γ-³²P-ATP in a suitable buffer system, and measuring the incorporation of ³²P.

Methods for Assessing the Efficacy of Nek2 inhibitors

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit Nek2 gene expression or biological activity. As discussed in further detail in the Examples provided below, Nek2 inhibitors of the present invention are effective in reducing Nek2 expression levels. Thus, the present invention further discloses methods that permit the skilled artisan to assess the effect of candidate inhibitors.

Candidate Nek2 inhibitors may be tested by administration to cells that either express endogenous Nek2 or that are made to express Nek2 by transfection of a mammalian cell with a recombinant Nek2 plasmid construct.

Effective Nek2 inhibitory molecules will be effective in reducing the levels of Nek2 mRNA as determined, e.g., by Northern blot or RT-PCR analysis. For a general description of these procedures, see, e.g., Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pasternak, J. J. 1998) incorporated herein by reference. The effectiveness of a given candidate antisense molecule may be assessed by comparison with a control “antisense” molecule known to have no substantial effect on Nek2 expression when administered to a mammalian cell. Exemplary control molecules include the Nek2 oligonucleotides of SEQ ID NOs:13-22.

In alternate embodiments of the present invention, the effect of Nek2 inhibitors on the rate of DNA synthesis after challenge with a radiation or chemotherapeutic agent may be assessed by, e.g., the method of Young and Painter. Hum. Genet. (1989) 82:113-117. Briefly, culture cells maybe incubated in the presence of ¹⁴C-thymidine prior to exposure to, e.g., X-rays. Immediately after irradiation, cells are incubated for a short period prior to addition of ³H-thymidine. Cells are washed, treated with perchloric acid and filtered (Whatman GF/C). The filters are rinsed with perchloric acid, 70% alcohol and then 100% ethanol; radioactivity is measured and the resulting ³H/¹⁴C ratios used to determine the rates of DNA synthesis.

Nek2 inhibitors effective in reducing Nek2 gene expression by one or more of the methods discussed above may be further characterized in vivo for efficacy in one of the readily available animal model systems. Various animal model systems for study of cancer and genetic instability associated genes are disclosed in, for example, Donehower, L. A. Cancer Surveys (1997) 29:329-352 incorporated herein by reference.

Pharmaceutical Compositions

The antisense oligonucleotides and ribozymes of the present invention can be synthesized by any method known in the art for ribonucleic or deoxyribonucleic nucleotides. For example, the oligonucleotides can be prepared using solid-phase synthesis such as in an Applied Biosystems 380B DNA synthesizer. Final purity of the oligonucleotides is determined as is known in the art.

The antisense oligonucleotides identified using the methods of the invention modulate tumor cell proliferation. Therefore, pharmaceutical compositions and methods are provided for interfering with cell proliferation, preferably tumor cell proliferation, comprising contacting tissues or cells with one or more of antisense oligonucleotides identified using the methods of the invention. Preferably, an antisense oligonucleotide having one of SEQ ID NOs:3-12 is administered.

The methods and compositions may also be used to treat proliferative disorders including other forms of cancer such as leukemias, lymphomas (Hodgkins and non-Hodgkins), sarcomas, melanomas, adenomas, carcinomas of solid tissue, hypoxic tumors, squamous cell carcinomas of the mouth, throat, larynx, and lung, genitourinary cancers such as cervical and bladder cancer, hematopoietic cancers, colon cancer, pancreatic cancer, head and neck cancers, and nervous system cancers, benign lesions such as papillomas, arthrosclerosis, psoriasis, primary and secondary polythemia, mastocytosis, autoimmune diseases, angiogenesis, bacterial infections, and viral infections, such as HIV infections, hepatitis or herpes infections.

The invention provides pharmaceutical compositions of antisense oligonucleotides and ribozymes complementary to the Nek2 mRNA gene sequence as active ingredients for therapeutic application. These compositions can also be used in the method of the present invention. Where required the compounds are nuclease resistant. In general the pharmaceutical composition for modulating cell proliferation or for cytotoxicity in a mammal includes an effective amount of at least one antisense oligonucleotide as described above needed for the practice of the invention, or a fragment thereof shown to have the same effect, and a pharmaceutically physiologically acceptable carrier or diluent.

In one embodiment of the invention, a method is provided for reducing metastasis in a subject comprising administering an amount of an antisense oligonucleotide of the invention effective to reduce metastasis. Most preferably the antisense oligonucleotide is one of SEQ ID NOs:3-12.

The pharmaceutical composition for inhibiting tumorigenicity of neoplastic cells in a mammal consists of an effective amount of at least one active ingredient selected from antisense oligonucleotides complementary to the Nek2 mRNA, including the entire Nek2 mRNA or having short sequences as set forth in SEQ ID NOs:3-12 and a pharmaceutically physiologically acceptable carrier or diluent. Combinations of the active ingredients can be used.

The compositions can be administered orally, subcutaneously or parenterally including intravenous, intraarterial, intramuscular, intraperitoneally, and intranasal administration as well as intrathecal and infusion techniques as required by the malignant cells being treated. For delivery within the CNS intrathecal delivery can be used with for example an Ommaya reservoir or other methods known in the art. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate oligonucleotide uptake. Implants of the compounds are also useful. In general, the pharmaceutical compositions are sterile.

In the method of the present invention, proliferating cells including neoplastic cells are contacted with a growth-inhibiting amount of the bioactive antisense oligonucleotide for the Nek2 mRNA or a fragment thereof shown to have substantially the same effect. In an embodiment, the mammal to be treated is human but other mammalian species can be treated in veterinary applications.

By bioactive (expressible) is meant that the oligonucleotide is biologically active in the cell when delivered directly to the cell and/or is expressed by an appropriate promotor and active when delivered to the cell in a vector as described below. Nuclease resistance is provided by any method known in the art that does not substantially interfere with biological activity as described herein.

“Contacting the cell” refers to methods of exposing or delivery to a cell of antisense oligonucleotides whether directly or by viral or non-viral vectors and where the antisense oligonucleotide is bioactive upon delivery. The method of delivery will be chosen for the particular cancer being treated. Parameters that affect delivery can include the cell type affected and tumor location as is known in the medical art.

The treatment generally has a length proportional to the length of the disease process and drug effectiveness and the patient species being treated. It is noted that humans are treated generally longer than the Examples exemplified herein, which treatment has a length proportional to the length of the disease process and drug effectiveness. The doses may be single doses or multiple doses as determined by the medical practitioners and treatment courses will be repeated as necessary until diminution of the disease is achieved. Optimal dosing schedules may be calculated using measurements of drug accumulation in the body. Practitioners of ordinary skill in the art can readily determine optimum dosages, dosing methodologies, and repetition rates. Optimum dosages may vary depending on the relative potency of the antisense oligonucleotide, and can generally be determined based on values in in vitro and in vivo animal studies and clinical trials. Variations in the embodiments used may also be utilized. The amount must be effective to achieve improvement including but not limited to decreased tumor growth, or tumor size reduction or to improved survival rate or length or decreased drug resistance or other indicators as are selected as appropriate measures by those skilled in the art.

Although some antisense oligonucleotides may not completely abolish tumor cell growth in vitro, these antisense compounds may be clinically useful if they inhibit tumor growth enough to allow complementary treatments, such as chemotherapy, to be effective. The pharmaceutical compositions of the present invention therefore are administered singly or in combination with other drugs, such as cytotoxic agents, immunotoxins, alkylating agents, anti-metabolites, antitumor antibiotics and other anti-cancer drugs and treatment modalities that are known in the art. The composition is administered and dosed in accordance with good medical practice taking into account the clinical condition of the individual patient, the site and method of administration, scheduling of administration, and other factors known to medical practitioners. The “effective amount” for growth inhibition is thus determined by such considerations as are known in the art. The pharmaceutical composition may contain more than one embodiment of the present invention.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promotor to allow the sequence to be expressed in the targeted cell.

Once the oligonucleotide sequences are ready for delivery they can be introduced into cells as is known in the art. Transfection, electroporation, fusion, liposomes, colloidal polymeric particles and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

The present invention provides vectors comprising an expression control sequence operatively linked to the oligonucleotide sequences of the invention. The present invention further provides host cells, selected from suitable eucaryotic and procaryotic cells, which are transformed with these vectors as necessary. Such transformed cells allow the study of the function and the regulation of malignancy and the treatment therapy of the present invention.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either procaryotic or eucaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al., BioTechniques (1986) 4:504-512 and include, for example, stable or transient transfection, lipofection, electroporation and infection with recombinant viral vectors.

Recombinant methods known in the art can also be used to achieve the antisense inhibition of a target nucleic acid. For example, vectors containing antisense nucleic acids can be employed to express an antisense message to reduce the expression of the target nucleic acid and therefore its activity.

The present invention also provides a method of evaluating if a compound inhibits transcription or translation of an Nek2 gene and thereby modulates (i.e., reduces) cell proliferation comprising transfecting a cell with an expression vector comprising a nucleic acid sequence encoding Nek2, the necessary elements for the transcription or translation of the nucleic acid; administering a test compound; and comparing the level of expression of the Nek2 with the level obtained with a control in the absence of the test compound.

The present invention provides detectably labeled oligonucleotides for imaging Nek2 polynucleotides within a cell. Such oligonucleotides are useful for determining if gene amplification has occurred, and for assaying the expression levels in a cell or tissue using, for example, in situ hybridization as is known in the art.

While the present invention has been described with specificity in accordance with certain of its preferred embodiments, the following examples serve only to illustrate the invention and are not intended to limit the invention.

### EXAMPLES

### Example 1

Antisense Inhibition of Target RNA

A. Preparation of Oligonucleotides for Transfection

A carrier molecule, comprising either a lipitoid or cholesteroid, was prepared for transfection by diluting to 0.5 mM in water, followed by sonication to produce a uniform solution, and filtration through a 0.45 μm PVDF membrane. The lipitoid or cholesteroid was then diluted into an appropriate volume of OptiMEM™ (Gibco/BRL) such that the final concentration would be approximately 1.5-2 nmol lipitoid per μg oligonucleotide.

Antisense and control oligonucleotides were prepared by first diluting to a working concentration of 100 μM in sterile Millipore water, then diluting to 2 μM (approximately 20 mg/mL) in OptiMEM™. The diluted oligonucleotides were then immediately added to the diluted lipitoid and mixed by pipetting up and down.

Antisense oligonucleotides are shown in SEQ ID NOs: 3-12. The corresponding reverse control oligonucleotides are:

| | |
|---|---|
| AAGTCAATGT CCTCGCTCTC GCTCG | (SEQ ID NO:13) |
| ATCCTCTCGG TCTTTTTAGC GTCCT | (SEQ ID NO:14) |
| ACGTAGCCCT AGAATTTGGT CGGTT | (SEQ ID NO:15) |
| CTGCTTCACT ACCACCAGTA TGGCA | (SEQ ID NO:16) |
| ACCAGTAACC CGACGAACGA CATAC | (SEQ ID NO:17) |
| CCTTACGGTG TCTGCTTCAC TACCA | (SEQ ID NO:18) |
| GGCCCGACTC CTGATACTTC ACAAC | (SEQ ID NO:19) |
| TCGTCTCCTG TTTGACCGAT CTCGT | (SEQ ID NO:20) |
| TCTTCTCCCG CTGTTAATCC TCTCG | (SEQ ID NO:21) |
| AAAGGACCTA CCGTTCGTTT TGCAG | (SEQ ID NO:22) |

B. Transfection

SW620 or MRC9 (normal fibroblast) cells were plated in growth media with serum at 2×10⁵ cells per well in 6-well culture dishes, and allowed to incubate overnight. The cells were then transfected by adding the oligonucleotide/lipitoid mixture, immediately after mixing, to a final concentration of 200 nM oligonucleotide. The cells were then incubated with the transfection mixture overnight at 37° C., 5% CO₂. After incubation, the transfection mixture was removed and replaced with normal growth media containing serum.

C. Total RNA Extraction and Reverse Transcription

Total RNA was extracted from the transfected cells using the RNeasy™ kit (Qiagen Corporation, Chatsworth, Calif.), following protocols provided by the manufacturer. Following extraction, the RNA was reverse-transcribed for use as a PCR template. Generally 0.2-1 μg of total extracted RNA was placed into a sterile microfuge tube, and water was added to bring the total volume to 12.5 μL. 7.5 μL of a buffer/enzyme mixture was added to each tube. The buffer/enzyme mixture was prepared by mixing, in the order listed, 2.5 μL H₂O, 2.0 μL 10× reaction buffer, 10 μL (20 pmol) oligo dT, 1.0 μL dNTP mix (10 mM each), 0.5 μL (20 u) RNAsin® (Ambion, Inc., Hialeah, Fla.) and 0.5 μL (50 u) MMLV reverse transcriptase (Ambion, Inc.). The contents of the microfuge tube were mixed by pipetting up and down, and the reaction was incubated for 1 hour at 42° C.

D. PCR Amplification and Quantification of Target Sequences

Following reverse transcription, target genes were amplified using the Roche Light CyclerTM real-time PCR machine. 20 μL aliquots of PCR amplification mixture were prepared by mixing the following components in the order listed: 2 μL 10× PCR buffer II (containing 10 mM Tris pH 8.3 and 50 mM KCl, Perkin-Elmer, Norwalk, Conn.) 3 mM MgCl₂, 140 μM each dNTP, 0.175 pmol of each Nek2 oligo, 1:50,000 dilution of SYBR® Green, 0.25 mg/mL BSA, 1 unit Taq polymerase, and H₂0 to 20 μL. SYBR® Green (Molecular Probes, Eugene, Oreg.) is a dye that fluoresces when bound to double-stranded DNA, allowing the amount of PCR product produced in each reaction to be measured directly. 2 μL of completed reverse transcription reaction was added to each 20 μL aliquot of PCR amplification mixture, and amplification was carried out according to standard protocols.

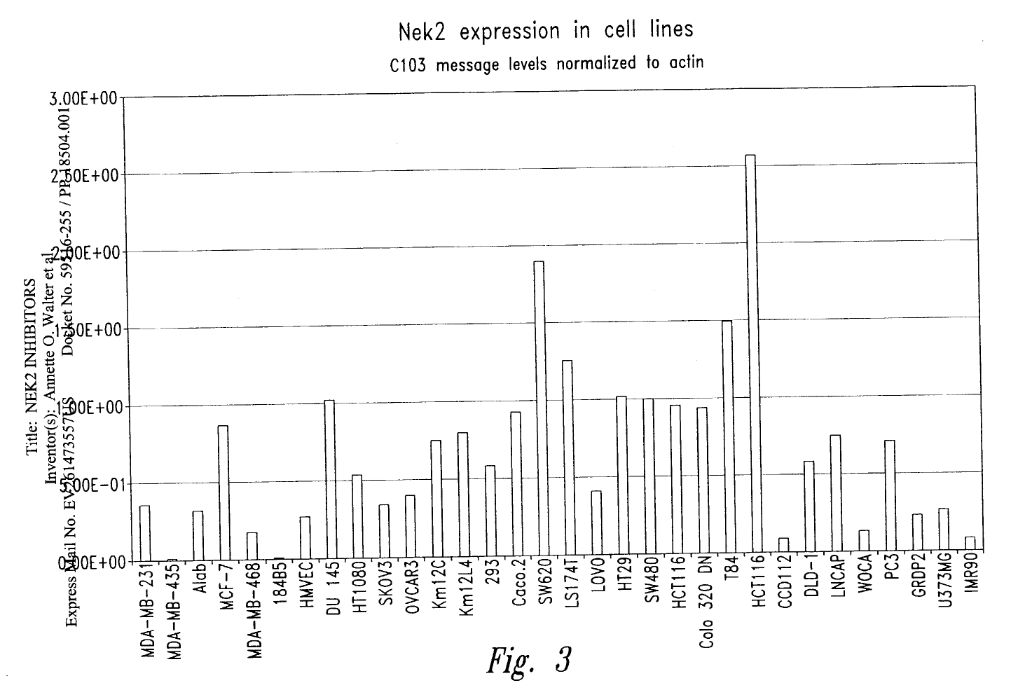

Amounts of amplified target sequences obtained from each PCR reaction were normalized through comparison with an internal control (e.g., beta-actin). FIG. 3 shows the relative levels of Nek2 message in a variety of cell lines, normalized to actin.

### Example 2

Soft Agar Assay

The bottom layer consisted of 2 ml of 0.6% agar in media plated fresh within a few hours of layering on the cells. For the cell layer, cells (SW620 and MRC9) transfected as described in Example 1 were removed from the plate in 0.05% trypsin and washed twice in media. Cells were counted in a coulter counter, and resuspended to 10⁶ cells per ml in media. 10 ml aliquots were placed with media in 96-well plates (to check counting with WST1), or diluted further for soft agar assay. 2000 cells were plated in 800 ml 0.4% agar in duplicate wells above 0.6% agar bottom layer.

Media layer: After the cell layer agar solidifies, 2 ml of media was bled on top and antisense or reverse control oligo was added without delivery vehicles. Fresh media and oligos are added every 3-4 days.

Colonies were formed in 10 days to 3 weeks. Fields of colonies were counted by eye. WST-1 metabolism values were used to compensate for small differences in starting cell number. Larger fields can be scanned for visual record of differences. SW620 cells transfected with antisense gave rise to fewer colonies compared to cells transfected with the control oligonucleotide.

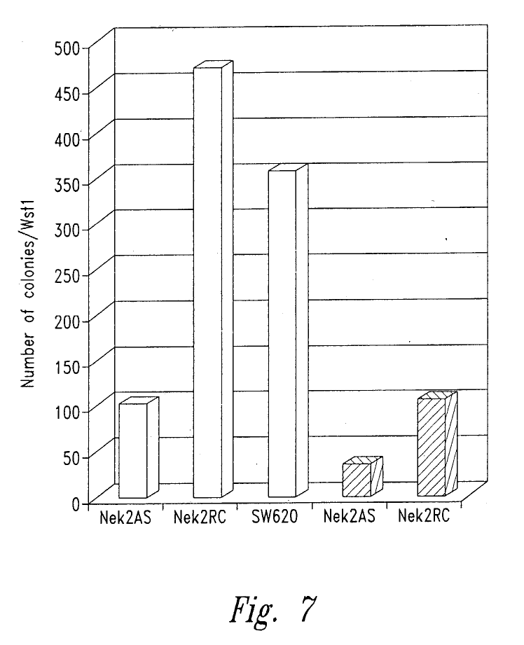

Cell colonies were counted in 6 randomly-selected grids across each soft-agar well. The number of colonies was normalized by comparison with a starting WST1 value, as shown in FIG. 7. (WST-1 Cell Proliferation Assay available from, for example, Panvera.)

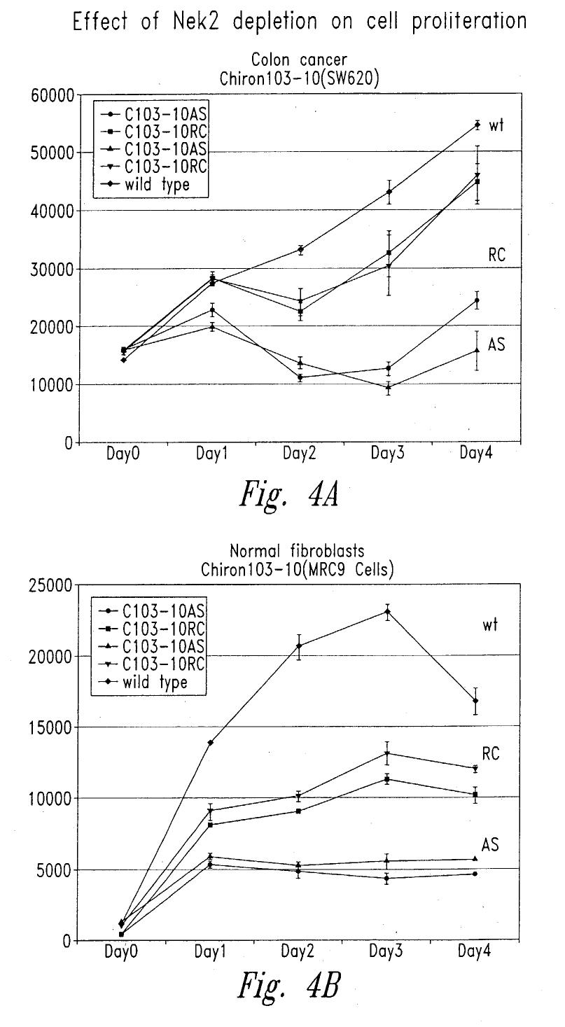

FIG. 4A indicates that treating SW620 cells with the Nek2 antisense oligonucleotide of SEQ ID NO:12 reduced cell proliferation rates, relative to control cells transfected with the respective reverse complement oligonucleotide. FIG. 4B shows the results of the same experiment using MRC9 cells.

### Example 3

Increased Release of Lactate Dehydrogenase by Antisense-Treated Cells

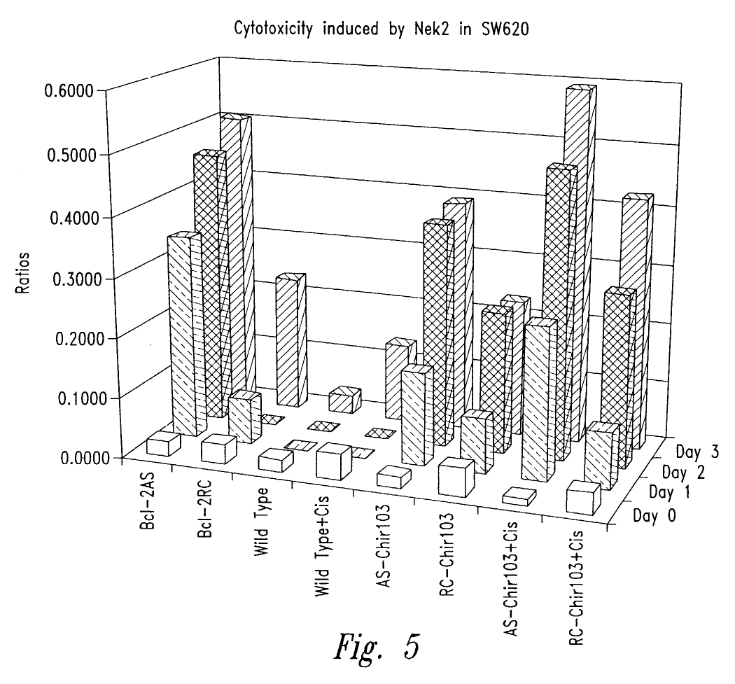

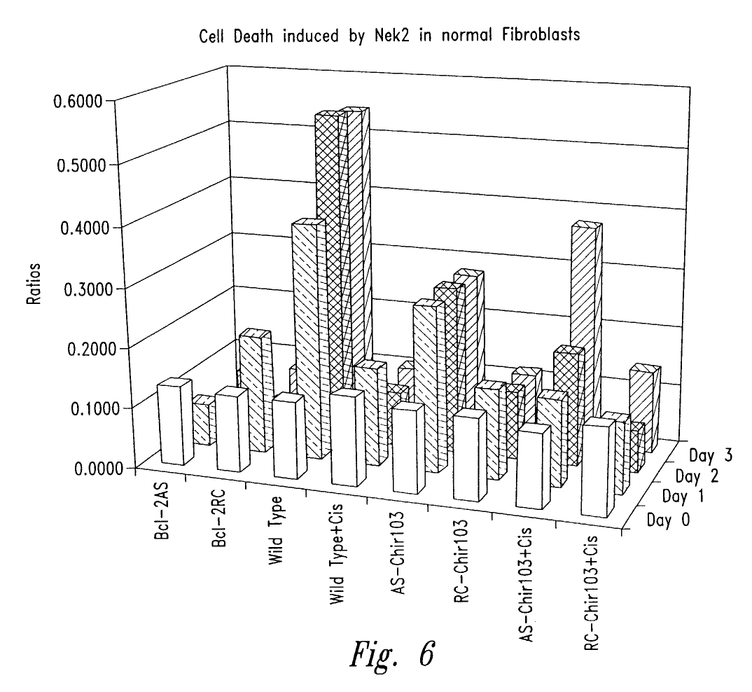

SW620 cells were transformed as described in Example 1 using the antisense oligonucleotide of SEQ ID NO:12 and the reverse complement. The LDH assay was performed using a Roche LDH kit. As shown in FIG. 5, SEQ ID NO:12 induced greater cytotoxicity than did the corresponding reverse control oligonucleotide. FIG. 6 shows that treatment of normal fibroblasts with SEQ ID NO:12 induced cell death in normal fibroblasts. Wild type cells are untransfected cells. “Wild type+Cis” refers to untransfected cells in the presence of cisplatin, to determine whether Nek2 inhibition is sensitizing for cisplatin. Bcl-2AS and Bcl-2RC are positive control oligonucleotides to validate the experimental conditions. It is known that inhibition of the anti-apoptotic protein Bcl-2 causes cells to enter apoptosis.

### Example 4

Nek2 mRNA Levels in Breast Cancer Tissue Samples

Nek2 mRNA levels are higher in 43% of breast cancer samples, using EVD (expression validation data). Further microarray analysis is performed as follows: Various human cDNA clones, including Nek2, are spotted onto microarray slides, with each spot representing one clone. The slides are hybridized with cDNA from tumor tissues of breast cancer patients, and the cDNA is labeled with a dye. The intensity of the signal is compared with the signal with cDNA of normal breast tissue.

From the foregoing it will be appreciated that, although specific embodiments of the invention have been described herein for purposes of illustration, various modifications may be made without departing from the spirit and scope of the invention. Accordingly, the invention is not limited except as by the appended claims.

1. An isolated Nek2 inhibitor selected from the group consisting of an anti sense oligonucleotide, a ribozyme, a protein, a polypeptide, an antibody, and a small molecule.

2. The isolated Nek2 inhibitor of claim 1 wherein said inhibitor is an antisense molecule.

3. The isolated Nek2 inhibitor of claim 2 wherein said inhibitor is an antisense molecule or the complement thereof comprising at least 10 consecutive nucleic acids of the sequence of SEQ ID NO: 1 or 23.

4. The isolated Nek2 inhibitor of claim 2 wherein said antisense molecule or the complement thereof hybridizes under high stringency conditions to the sequence of SEQ ID NO: 1 or 23.

5. The isolated Nek2 inhibitor of claim 2 wherein said antisense molecule comprises a nucleic acid sequence selected from the group consisting of SEQ ID NOs:3-12.

6. The isolated Nek2 inhibitor of claim 1 wherein said inhibitor is a ribozyme.

7. The isolated Nek2 inhibitor of claim 1 wherein said inhibitor is selected from the group consisting of an antibody and an antibody fragment.

8. A composition comprising a therapeutically effective amount of a Nek2 inhibitor in a pharmaceutically acceptable carrier.

9. The composition of claim 8, wherein said composition comprises two or more Nek2 inhibitors in the composition, and the Nek2 inhibitor is an antisense molecule.

10. The composition of claim 9, wherein the antisense molecule or the complement thereof comprises at least 10 consecutive nucleic acids of the sequence of SEQ ID NO:1 or 23.

11. The composition of claim 9, wherein the antisense molecule comprises a nucleic acid sequence selected from the group consisting of SEQ ID NOs:3-12.

12. A method of inhibiting the expression of Nek2 in a mammalian cell, comprising administering to said cell an Nek2 inhibitor selected from the group consisting of an antisense oligonucleotide, a ribozyme, a protein, a polypeptide, an antibody, and a small molecule.

13. The method of claim 12, wherein said Nek2 inhibitor is an antisense molecule.

14. A method of inhibiting the expression of Nek2 gene expression in a subject, comprising administering to said subject, in a pharmaceutically effective vehicle, an amount of an antisense oligonucleotide which is effective to specifically hybridize to all or part of a selected target nucleic acid sequence derived from said Nek2 gene.

15. The method of claim 14, wherein the antisense oligonucleotide is selected from the group consisting of SEQ ID NOs:3-12.

16. A method of treating neoplastic disease, comprising administering to a mammalian cell an Nek2 inhibitor selected from group consisting of an antisense oligonucleotide, a ribozyme, a protein, a polypeptide, an antibody, and a small molecule such that the neoplastic disease is reduced in severity.

17. An antisense oligonucleotide 8 to 35 nucleotides in length targeted to a nucleic acid molecule encoding human Nek2, wherein said antisense compound inhibits the expression of human Nek2.

18. An isolated oligonucleotide comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOs:3-12.

19. A recombinant vector comprising a polynucleotide, wherein said polynucleotide comprises a transcriptional initiation region and a sequence encoding an antisense oligonucleotide at least 8 nucleotides or nucleotide analogues and not longer than 35 nucleotides in length, wherein said antisence oligonucleotide comprises a sequence selected from the group consisting of SEQ ID NOs:3-12.

20. A polynucleotide probe for identifying Nek2 polynucleotides by in situ hybridization, wherein said probe comprises an oligonucleotide of selected from the group consisting of SEQ ID NOs:3-12, wherein said probe is detectably labeled.

21. A method to inhibit expression of a Nek2 target gene in a cell in vitro comprising introduction of a ribonucleic acid (RNA) into the cell in an amount sufficient to inhibit expression of the Nek2 target gene, wherein the RNA is a double-stranded molecule with a first strand consisting essentially of a ribonucleotide sequence which corresponds to a nucleotide sequence of the Nek2 target gene and a second strand consisting essentially of a ribonucleotide sequence which is complementary to the nucleotide sequence of the Nek2 target gene, wherein the first and the second ribonucleotide strands are separate complementary strands that hybridize to each other to form said double-stranded molecule, and the double-stranded molecule inhibits expression of the target gene.

22. The method of claim 21 in which the first ribonucleotide sequence comprises at least 25 bases which correspond to the Nek2 target gene and the second ribonucleotide sequence comprises at least 25 bases which are complementary to the nucleotide sequence of the Nek2 target gene.

23. The method of claim 21 in which the target gene expression is inhibited by at least 10%.

24. The method of claim 21 in which said double-stranded ribonucleic acid structure is at least 25 bases in length and each of the ribonucleic acid strands is able to specifically hybridize to a deoxyribonucleic acid strand of the Nek2 target gene over the at least 25 bases.

25. The method of claim 21 in which the expression of the Nek2 target gene is inhibited by at least 10%.

26. The method of claim 21 in which the RNA is introduced within a body cavity of the organism and outside the target cell.

27. The method of claim 21 in which the RNA is introduced by extracellular injection into the organism.

28. The method of claim 21 in which the organism is contacted with the RNA by feeding the organism food containing the RNA.

29. The method of claim 28 in which the food comprises a genetically-engineered host transcribing the RNA.

30. The method of claim 21 in which at least one strand of the RNA is produced by transcription of an expression construct.
